(19) United States
(12) Patent Application Publication
(10) Pub. No.: US 2016/0074483 A1
(43) Pub. Date: Mar. 17, 2016

(54) THERAPEUTIC PEPTIDE-EXPRESSING CELLS

(71) Applicant: INFINITE CELLS, LLC, Alameda, CA (US)

(72) Inventor: David Kiewlich, Alameda, CA (US)

(73) Assignee: Infinite Cells, LLC, Alameda, CA (US)

(21) Appl. No.: 14/783,416

(22) PCT Filed: Apr. 11, 2014
(86) PCT No.: PCT/US2014/033871

§ 371 Date: Oct. 8, 2015

Related U.S. Application Data
(60) Provisional application No. 61/811,576, filed on Apr. 12, 2013.

(51) Int. Cl. A61K 38/37

(52) U.S. Cl. CPC A61K 38/37; A61K 48/00

(57) ABSTRACT

Provided herein are cells for in vivo expression of therapeutic proteins or peptides, methods of making and using the same.

### FIELD OF THE INVENTION

This invention is directed to protein- or peptide-expressing stem cells, methods of making and using such protein- or peptide-expressing stem cells, particularly for treatment of conditions in a subject.

### BACKGROUND OF THE INVENTION

It has been known in the art to administer a purified protein or peptide to treat a subject suffering from a certain condition, such as cancer, hypertension, chronic pain, etc. However, the therapeutic effects may be limited due to various factors such as: the requirement of repeated or frequent administration, especially for long-term therapy, degradation of protein or peptide before reaching the target site, and immunogenicity of foreign proteins/peptides. Even when such factors are addressed, there remains other factors such as the frequency and amount of dosing of the therapeutic protein or peptide as the peak-to-trough concentration must be taken into account. This, of course, will vary from patient to patient as well as within a given patient based on the time of day, fasting or fed condition, the degree and severity of the illness to be treated. All of these will vary due to many other contributing factors, all of which need to be taken into consideration. It would be ideal to provide a steady amount of the therapeutic protein or peptide to a subject over a prolonged period of time without repeated administration or causing immune responses (e.g., rejection).

Conventionally, cell therapy treats a subject by transplanting expanded cells into a subject such that the subject has a sufficient amount of the cells due to in vivo self-renewal of the transplanted cells. Typically, embryonic stem cells and adult stem cells are used in cell therapy. See, for example, Genetic Engineering and Biotechnology News, “FDA Clears Geron to Start World's First Trial with hESC Therapy,” Jul. 30, 2010 (available at http://www.genengnews.com/gen-news-highlights/fda-clears-geron-to-start-world-s-first-trial-with-hesc-therapy/81243731/); and Rama, et al., N Engl. J. Med. 363(2):147-155 (2010). Alternatively, it was reported that stem cells were modified to produce a needed substance in vivo. See Cavazzana-Calvo, et al., Nature 467:318-322 (2010). Nevertheless, conventional cell therapy focuses on using the cells rather than proteins or peptides as a therapy. Additionally, even when a specific protein is expressed, the protein would be the wild type version of the protein.

Therefore, there is a need in the art to develop a method for administering a therapeutically effective amount of a protein or a peptide to a subject for a prolonged period of time as needed while avoiding disadvantages of the therapy, such as the requirement for repeated administration, rapid degradation of the protein or peptide, or immunogenicity.

### SUMMARY OF THE INVENTION

Provided herein is a methodology for treating a condition mediated at least in part by the absence or incomplete expression of a protein or peptide in a subject by providing a protein or a peptide to a subject in a therapeutically effective amount for a prolonged period of time. The method entails implanting genetically modified stem cells to a subject such that the stem cells express a therapeutic amount of the protein or peptide in vivo.

In one aspect, this invention is directed to stem cells expressing a therapeutically effective amount of a protein or a peptide in vivo upon implantation into a subject. In one embodiment, the stem cells are not in an active expansion phase. In another embodiment, the stem cells are derived from or isolated from an adipose tissue. Preferably, the stem cells are autologous stem cells isolated from the subject to be treated.

In another aspect, this invention provides an isolated autologous, adipose stem cell which has been modified to express a protein or peptide which is preferably endogenous to a subject from which the stem cell was isolated. In some embodiments, expression of the protein or peptide by the stem cell may require a triggering step such as a feedback loop where expression is initiated by a defined lower concentration of the protein or peptide and expression is terminated by a defined higher concentration of the protein or peptide.

In another aspect, the invention relates to a method for treating a disease or condition mediated at least in part by the absence or insufficient expression of a protein or a peptide in a subject. The method has the following steps: (a) isolating autologous stem cells from the patient; (b) modifying the stem cells so as to express the protein or the peptide; and (c) providing a sufficient population of the modified stem cells in the patient which express in the aggregate a therapeutic concentration of the protein or the peptide in vivo to treat the disease or condition.

In a related aspect, the invention is directed to a method for delivering a protein or a peptide to a subject. The method has the following steps: (a) isolating autologous stem cells from the patient; (b) modifying the stem cells so as to express the protein or the peptide; and (c) providing the modified stem cells in the patient so as to express the protein or the peptide in vivo.

The proteins or peptides encompassed by this invention are either naturally occurring or synthetic. Preferably, the proteins or peptides are modified to facilitate penetration across blood brain barrier, to reduce degradation before reaching the target site, to reduce immunogenicity, and/or to increase efficacy or potency upon in vivo expression in the subject.

### BRIEF DESCRIPTION OF THE DRAWINGS

The accompanying drawing(s), which are incorporated in and constitute a part of this specification, illustrate several aspects described below.

FIG. 1 is an overview flow chart of the process of a cell therapy.

### DETAILED DESCRIPTION OF THE INVENTION

It is to be understood that this invention is not limited to particular embodiments described, as such may vary. It is also to be understood that the terminology used herein is for the purpose of describing particular aspects and embodiments only, and is not intended to be limiting the scope of this invention.

It must be noted that as used herein and in the appended claims, the singular forms “a”, “an”, and “the” include plural referents unless the context clearly dictates otherwise.

### Definitions

Unless defined otherwise, all technical and scientific terms used herein have the same meaning as commonly understood by one of ordinary skill in the art to which this invention belongs. As used herein, the following terms have the following meanings.

The term “about” when used before a numerical designation, e.g., temperature, time, amount, and concentration, including range, indicates approximations which may vary by (+) or (−) 10%, 5% or 1%. One skilled in the art would understand the approximation associated with a specific value or range.

As used herein, the term “pharmaceutically acceptable” refers to safe and non-toxic for in vivo, preferably human, administration.

As used herein, the term “therapeutically effective amount” refers to the amount of a protein or a peptide expressed according to this invention that is sufficient to effect treatment, as defined herein, when administered to a subject in need of such treatment. The therapeutically effective amount will vary depending upon the subject and condition being treated, the weight and age of the subject, the severity of the condition, the particular composition or excipient chosen, the dosing regimen to be followed, timing of administration, the manner of administration and the like, all of which can be determined readily by one of ordinary skill in the art.

As used herein, the term “treatment” or “treating” means any treatment of a disease or condition in a patient, including:
preventing or protecting against the disease or condition, that is, causing the clinical symptoms not to develop, for example, in a subject at risk of suffering from such a disease or condition, thereby substantially averting onset of the disease or condition;
inhibiting the disease or condition, that is, arresting or suppressing the development of clinical symptoms; and/or
relieving the disease or condition, that is, causing the regression of clinical symptoms.

The terms “patient” and “subject” are used interchangeably, referring to mammals and including humans and non-human mammals.

The terms “protein” and “peptide” are sometimes used interchangeably in this application. Both protein and peptide comprise a continuous sequence of amino acids joined covalently by peptide bonds. The main difference between a protein and a peptide is the size, where a peptide contains 50 amino acids or less, and a protein contains more than 50 amino acids. Conventionally, a protein is defined as a functional, polypeptide chain composed of at least 50 amino acids.

Cells Expressing a Protein or a Peptide in vivo

The invention is directed to treating a condition mediated at least in part by the absence or insufficient expression of a protein or a peptide in a patient. Specifically, genetically engineered stem cells are used as a device for long term expression of a protein or a peptide such that the stem cells effectively act as an infinite depot for continuous expression of the protein or the peptide in vivo over an extended period of time. Thus, the invention provides not only a long term therapy for a disease associated with the lack or insufficiency of a protein or a peptide but also a mechanism to deliver a therapeutic protein or peptide for treating diseases such as cancer, anemia and the like, thereby eliminating the need for frequent infusions.

In one aspect, the cells used in this invention are not in an active expanding phase and have low tumor forming potential in vivo once administered to a subject. In one embodiment, exemplary cells as determined by the sources of the cells include, but are not limited to, adipose stem cells, mesenchymal stem cells, umbilical cord blood (UCB) stem cells, and somatic cells. In another embodiment, exemplary types of cells include, but are not limited to, adipose cells, endothelial cells, hepatocytes, and stem cells.

It is within the purview of one skilled in the art to choose a specific type of cells based on the balance of a number of factors, such as the risk of inducing tumor, the difficulties associated with genetic engineering, maintenance and differentiation of the cells, the expected in vivo life span of the cells, the engraftment potential, potential for inducing immunogenicity, and the target site for administration.

In one embodiment, adipose stem cells are used in this invention. Adipose stem cells are readily available with a long history of cosmetic use. U.S. Pat. No. 7,470,537, the content of which is incorporated herein by reference, describes certain uses of adipose stem cells. Currently, adipose stem cells are approved by the FDA to be cultured in anticipation for injection for cosmetic purposes such as body sculpting with no concern for transformation. These cells can be engineered at specific sites in the genome and then these integration sites can be readily identified, e,g., by genome DNA sequencing. These cells also can be readily verified for being nononcogenic through various in vivo or in vitro tests known in the art. For example, the cells can be grown in vitro to detect a loss of contact inhibition, which is an indication of transformation. Alternatively, the cells can implanted into mice in vivo to detect tumor formation in the mice.

The cells are genetically engineered to express a desired protein or peptide. In one embodiment, the cells are modified to establish an optimal inducible expression system such that the protein or peptide expression is under control. In another embodiment, the cells' expression system is modified to incorporate a “kill-switch” to destroy the therapeutic cells to effectively terminate the in vivo expression of the protein or peptide.

The inducible expression systems are discussed in scientific publications, e.g., Meyer-Ficca et al., “Comparative analysis of inducible expression systems in transient transfection studies,” Analytical Biochemistry 334(1):9-19 (2004), the content of which is incorporated by reference.

For instance, a metalothionine promoter can be activated by exposure to heavy metal such as high levels of zinc.

Other exemplary exogenous inducible systems include the tetracycline inducible system and the R×R steroid receptor system. The tet system uses the bacterially derived tet-binding protein to regulate the expression of a tet response element controlled gene, upon exposure to the tetracycline related compound doxycycline.

The R×R steroid receptor system uses the insect molting hormone eceptor to regulate the expression of an R×R response element controlled gene, upon exposure to the insect molting hormone or related synthetic ligands.

These exemplary exogenous inducible systems are reasonably well controlled because neither tet nor R×R proteins are naturally occurring in mammalian cells.

When the expression of the therapeutic protein or peptide is no longer desired, the expression can be effectively terminated by a “kill-switch.” Specifically, the cells expressing the therapeutic protein or peptide are further genetically engineered to express a switch-protein that is not functional in mammalian cells under normal physiological condition. Only upon administration of a drug that specifically targets this switch-protein, the cells expressing the switch-protein will be destroyed thereby terminating the expression of the therapeutic protein or peptide. Such a “kill-switch” system is known in the art, and therefore, it is within the purview of one skilled in the art to select and employ a suitable “kill-switch” system.

For instance, it was reported that cells expressing HSV-thymidine kinase can be killed upon administration of drugs, such as ganciclovir and cytosine deaminase. See, for example, Dey and Evans, Suicide Gene Therapy by Herpes Simplex Virus-1 Thymidine Kinase (HSV-TK), in Targets in Gene Therapy, edited by You (2011); and Beltinger et al., “Herpes simplex virus thymidine kinase/ganciclovir-induced apoptosis involves ligand-independent death receptor aggregation and activation of caspases,” Proc. Natl. Acad. Sci. USA 96(15):8699-8704 (1999).

In another aspect, proteins and peptides encompassed by this invention are modified to have certain properties better adapted for in vivo therapeutic effects. For instance, the protein or the peptide can be modified to penetrate the blood-brain harrier; to reduce the rate of degradation before reaching the target site; to reduce potential immunogenicity; to increase the specific enzymatic activity; to act as a specific inhibitor of a natural enzyme; to act as a decoy antigen for the immune system; to act as an antibiotic; to function as an antiperspirant or deodorant; to contain a lymphokine; to contain an inummoglobulin, an antiserum, an antibody, or fragment thereof; to contain an antigen, an epitope, or another immuno-specific immunoeffector that may be proteinaceous, to contain a nonspecific immunoeffector that may be proteinaceous; and/or to contain enzymes.

Ideally, adipose stem cells are genetically modified in vivo to express the desired therapeutic protein or peptide. For example, in vivo modification can be done via a virus vector that is modified to contain a ligand or receptor which binds with high specificity to a receptor or ligand on a specific cell type, e.g., adipose stem cells, or introduced into/onto that cell.

Methods for Making Cells Expressing a Protein or a Peptide in vivo

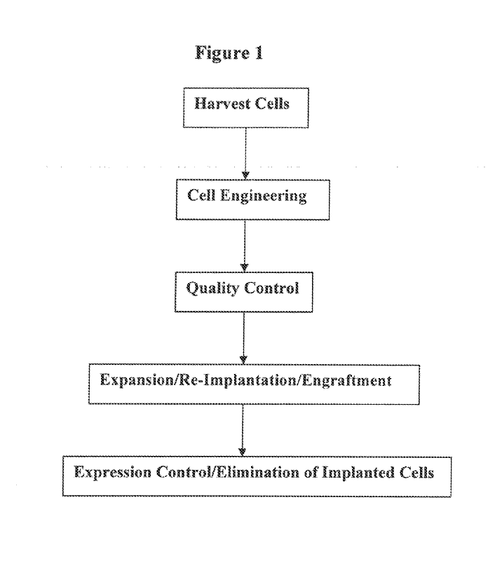

This invention also is related to modifying cells so as to express a protein or a peptide in vivo. FIG. 1 is an exemplary flow chart demonstrating the process of cell therapy. Briefly, the cells are isolated, preferably from a subject to receive the treatment. For example, adipose stem cells can be isolated by liposuction according to established procedure in the field of the art. Alternatively, certain cells such as adipose stem cells are commercially available.

The isolated cells are genetically engineered to insert a desired transgene encoding the protein or peptide at the target site in the genome. For quality control, the genome is sequenced to verify the accuracy of the sequence and the location of the transgene. Additionally, the engineered cells are tested for expression control and the proper function of the kill-switch.

Subsequently, the engineered cells are undergoing expansion, reimplantation and/or engraftment. For example, the modified adipose stem cells can be expanded, re-implanted and/or engrafted according to currently approved protocols developed for body sculpting.

Following engraftment, the level of the therapeutic protein or peptide is optimized through the use of an inducible expression system. Preferably, the level of the protein or peptide expressed in vivo correlates with the amount of the inducer administered to the subject. The efficacy of the therapy can be tested and demonstrated in animal models designed to have a specific condition or disease. If there are any undesired in vivo effects, the cell implant can be destroyed through activation of the kill-switch, thereby terminating the expression of the protein or peptide.

Uses of Cells Expressing a Protein or a Peptide in vivo

The genetically modified cells expressing a protein or a peptide in vivo have numerous medical applications, particularly useful for long-term therapy that requires a constant expression of the protein or peptide. The invention overcomes issues associated with conventional peptide therapy, such as eliminating the need for repeated, frequent administration or infusion of the protein or peptide, lowering the treatment cost, and minimizing immunogenicity by autologous production of the protein or peptide, etc.

More specifically, the invention can be used in treating a number of conditions or diseases, including but not limited to hypertension, congestive heart failure, diseases requiring anti-coagulant treatment, cancer, chronic pain, hyperuricemia and gout, and phenylketonuria (PKU).

Hypertension: Hypertension is a very common condition. Currently there are a number of oral drugs available, but they rely on the patient taking them regularly and they have a number of side effects. Once a patient starts hypertension therapy, the patient is likely to remain on the therapy for a prolonged period of time, even for the rest of the life of the patient. The invention provides a treatment by re-implanting or engrafting the patient's cells that are engineered to express an antibody or a fragment thereof that acts to increase vasodilation, which results in a reduction in blood pressure.

Congestive heart failure: Frequently, congestive heart failure is associated with hypertension. When the heart is forced to beat harder due to high blood pressure, it eventually gives out and fails. At the present, the only treatment for congestive heart failure is an artificial booster culminating in a heart transplant. A therapeutic protein, relaxin, may alleviate congestive heart failure. However, the use of relaxin is limited due to its poor circulating half-life, which makes it impractical for infusion. This invention provides a treatment that releases a therapeutic protein or peptide, such as relaxin, that would reduce the intensity of the heartbeat, thereby prolonging the time to a heart transplant. This is because the genetically modified cells allow continuous in vivo release of the protein to overcome the issue of short half-life of the protein.

Anti-coagulants: Anti-coagulants are another type of therapy that requires long-term administration. Anti-coagulants are used to treat or prevent a variety of diseases, including atrial fibrillation, deep vein thrombosis, pulmonary embolism, clotting disorders, stroke, heart attack, and adverse effects related to artificial heart valves. Currently, anti-coagulants derived from warfarin are being replaced with inhibitors of Factor X, which have better safety profiles. The present invention allows a very effective in vivo production of an inhibitor of Factor X without dependence on patient compliance. Inhibitors of Factor X may be naturally-occurring or synthetic, and include, without limitation, antistasin, tick anticoagulant peptide, and other anticoagulants derived from animal venoms (e.g., from centipedes, snakes, and the like).

Cancer: Monoclonal antibodies are commonly used to treat a wide variety of cancers. The monoclonal antibodies are typically synthesized in large fermentation tanks, purified and then infused into patients. Although monoclonal antibodies tend to specifically bind intended antigens, undesired cross-reactivity and side effects may occur when the antibodies are infused to a subject at a very high concentration. This invention allows continuous in vivo expression of the monoclonal antibodies at a therapeutically effective amount, thereby reducing or eliminating these undesired side effects.

Chronic Pain: Currently there are only two major options for treating chronic pain, nonsteroidal anti-inflammatory drugs (NSAIDs) and opiates; both have significant side effects. Pain is transmitted through the activity of a particular enzyme, COX-2. NSAIDs inhibit COX-2 and thus block pain. However, NSAIDs also inhibit COX-1 which is required for a number of homeostasis activities. Some side effects of NSAIDs are due to their cross reactivity with COX-1. Since protein inhibitors, such as antibodies or fragments thereof, can be designed to be highly specific, both by selection for affinity to COX-2 and by a lack of affinity to COX-1, these side effects can be significantly reduced or eliminated. Alternatively, a protein inhibitor can target a section of the COX-2 enzyme other than the active site, which may result in better specificity than a small molecule inhibitor which needs to target the active site due to the small size of the drug. As such, the present invention is more efficient in eliminating side effects than other small molecule inhibitors.

Opiates bind to a receptor in the central nervous system that controls a patient's ability to feel pain. The natural ligand for this opiate-receptor system does not seem to have any side effects but cannot be used therapeutically because of the ligand's limited circulating half-life. This invention allows continuous in vivo expression and release of the ligand to provide an effective therapy for chronic pain.

Hyperuricemia and gout: Hyperuricemia is characterized by abnormally high levels of uric acid in the blood. It can lead to gout, kidney stones, and kidney failure. This invention allows continuous in vivo expression of urate oxidase to convert uric acid to allantoin.

Phenylketonuria: Phenylketonuria (PKU) is a metabolic disorder wherein mutation of the phenylalanine hydroxylase gene causes loss of the ability to metabolize the amino acid phenylalanine (Phe) to tyrosine. PKU can result in intellectual disability, seizures, hyperactivity, and other serious medical conditions. When diagnosed in newborns, some or all of the clinical symptoms can be avoided or attenuated by strict diet and amino acid supplementation, generally throughout the patient's lifetime. This invention allows for in vivo expression of functional phenylalanine hydroxylase in a patient to regulate the levels of phenylalanine and treat PKU.

This invention is further defined by reference to the following example(s). It will be apparent to those skilled in the art that many modifications, both to materials and methods, may be practiced without departing from the scope of the current invention.

### EXAMPLE 1

### Protein Expression in Animal Models

### Materials

A murine model system, for example, HemA mice which do not express Factor VIII and are coagulation-deficient, is obtained from Jackson Labs. Murine adipose stem cells (mASC) are available for purchase from Lonza. Expression vectors, including expression vector for the protein or peptide of interest, and expression vector for a genetic “kill-switch” such as thymidine kinase fused to a selection marker blasticidin S resistence (TK-Blast), are available for purchase from Life Technologies. Factor VIII expression sequence is either cloned from human cDNA library or synthesized from Blue Heron or GenScript. TK-Blast cDNA is synthesized from Blue Heron or GenScript. Various lab equipment for molecular biology, protein purification and analysis is standard and known to one skilled in the art.

### Method

Factor VIII cDNA is inserted into the expression vector, and TK-Blast is inserted into a separate expression vector according to known protocols. The expression vectors are co-transfected into mASC and cells are selected for Blasticidin S resistance. Transfected mASC cells are “cloned” and expanded. Stem cells cannot be cloned from single cells, so individual colonies will consist of approximately 10 cells. The expression and function of Factor VIII in mASC cells are verified by Western Blotting, and in vitro functional test with HemA plasma, in 96-well format, which is available commercially. Optionally, large scale protein analysis is performed with mass spectrometry.

The “kill-switch” function is verified by treating transfected mASC cells with Ganciclovir and confirming that the cells die in the presence of Ganciclovir. Ganciclovir is non-toxic until enzymatically activated by thymidine kinase, a protein not endogenously expressed by mammals. Cells expressing TK should be sensitive to Ganciclovir, while parental mASC should not. Cell death can be assayed through a variety of ways, including Alomar Blue, Trypan Blue, or BrDU incorporation.

Two or three of the best Factor VIII expressing “clones” and two or three non-expressing “clones” are selected for further tests. Optionally, the whole genome of the Factor VIII-expressing cells are sequenced to identify the location of the expression constructs within the genome. The selected clones are sent to a contract research organization (CRO) for implantation into HemA mice.

The following experiments will be performed:

a dose response of implanted cell number to determine the optimal amount of cells to be implanted and to show that there is a cell dose correlation with expression level of the protein;

functional rescue of HemA challenges, such as tail clip, with Factor VIII expressing cell implant, but not with non-expressing cell implant;

experiments to show a functional “kill-switch” by the lack of expression of Factor VIII following treatment of the mice with Ganciclovir, by blood testing and/or functional testing; and

optionally, experiments to determine the protein expression by the implanted cells, which can be done by blood draws at predetermined, regular intervals to detect the Factor VIII expression levels and specific amounts over time.

It is within the purview of one skilled in the art, without undue experimentation, to select a different protein or peptide for the treatment of a different condition for optimizing expression in a suitable animal model.

### EXAMPLE 2

### Protein- or Peptide-Expressing Cells for Treating Humans

Adipose stem cells are extracted from a prospective patient and purified. The inducible expression vector is modified to insert the expression construct of the protein of interest such that the vector delivers the sequence encoding the protein of interest to a specific site in the genome through homologous recombination. Following selection and “cloning” similar to the procedure described in Example 1, the genome of individual clones are sequenced to verify the placement of the expression constructs within the genome. Subsequently, the adipose stem cells expressing the protein of interest are provided to the patient.

The contents of all reference(s), and patent(s), and patent application publication(s) cited in this application are incorporated by reference.

1. A method for treating a disease or a condition mediated by the absence or incomplete expression of a protein or a peptide in a patient, comprising:
(a) isolating autologous stem cells from said patient;
(b) modifying said stem cells so as to express said protein or peptide; and
(c) providing a sufficient population of said modified stem cells to said patient so as to express a sufficient amount of said protein or peptide in vivo to treat said disease or condition.

2. The method of claim 1, wherein said stem cells are adipose-derived stem cells.

3. The method of claim 1, wherein said stem cells are isolated from an adipose tissue.

4. The method of claim 1, wherein said protein or peptide is modified to facilitate penetration across blood brain barrier upon expression in said patient.

5. The method of claim 1, wherein said protein or peptide is modified to reduce degradation before reaching the target site upon expression in said patient.

6. The method of claim 1, wherein said protein or peptide is modified to reduce immunogenicity upon expression in said patient.

7. The method of claim 1, wherein said protein or peptide is modified to increase the efficacy or potency upon expression in said patient.

8. The method of claim 1, wherein the protein or peptide is chosen from the group consisting of a vasodilator, relaxin, a Factor X inhibitor, an antibody, an antibody fragment, an opioid receptor ligand, urate oxidase, and phenylalanine hydroxylase.

9. The method of claim 1 , wherein the disease or condition is chosen from the group consisting of hypertension, congestive heart failure, atrial fibrillation, deep vein thrombosis, pulmonary embolism, clotting disorders, stroke, heart attack, cancer, chronic pain, hyperuricemia, and phenylketonuria.

10. A method for delivering a protein or a peptide to a patient comprising:
(a) isolating autologous stem cells from said patient;
(b) modifying said stem cells so as to express said protein or peptide; and
(c) providing a sufficient population of said modified stem cells to said patient so as to express a sufficient amount of said protein or peptide in vivo in said patient.

11. The method of claim 10, wherein said protein or peptide is a naturally occurring protein or peptide or a synthetic protein or peptide.

12. The method of claim 10, wherein said stem cells are not in an active expansion phase.

13. The method of claim 10, wherein said stem cells are adipose-derived stem cells.

14. The method of claim 10, wherein the protein or peptide is chosen from the group consisting of a vasodilator, relaxin, a Factor X inhibitor, an antibody, an antibody fragment, an opioid receptor ligand, urate oxidase, and phenylalanine hydroxylase.

15. The method of claim 10, wherein the amount of said protein or peptide expressed in the patient is sufficient to treat a disease or a condition mediated by the absence or incomplete expression of a protein or a peptide in the patient.

16. The method of claim 16, wherein the disease or condition is chosen from the group consisting of hypertension, congestive heart failure, atrial fibrillation, deep vein thrombosis, pulmonary embolism, clotting disorders, stroke, heart attack, cancer, chronic pain, hyperuricemia, and phenylketonuria.
